(19) United States
(12) Patent Application Publication
(10) Pub. No.: US 2017/0151155 A1
(43) Pub. Date: Jun. 1, 2017

(54) HAIR TREATMENT AGENTS

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/366,278

(22) Filed: Dec. 1, 2016

(30) Foreign Application Priority Data
Dec. 1, 2015 (DE) 10 2015 223 816.4

(51) Int. Cl. A61K 8/73; A61K 8/42; A61K 8/43; A61K 8/46; A61K 8/64; A61Q 5/12; A61Q 5/02; A61K 8/60; A61K 8/19; A61K 8/34

(52) U.S. Cl. CPC A61K 8/737; A61K 8/19; A61K 8/42; A61K 8/731; A61K 8/43; A61K 8/345; A61K 8/64; A61Q 5/12; A61Q 5/02; A61K 8/60; A61K 8/466; A61K 2800/596; A61K 2800/594; A61K 2800/5426

(57) ABSTRACT

Hair treatment agents including: at least one anionic surfactant from the group of alkyl sulfates and/or alkyl ether sulfates; at least one amphoteric and/or nonionic surfactant; at least one divalent or trivalent metal salt; at least one cationic polymer; and ethyl lauroyl arginate.

### BACKGROUND

The present invention relates to hair treatment agents. In particular, to shampoos and conditioners having active ingredients for hair care.

The importance of hair care products with the longer performance has grown. This is due in part to increased stress on hair, for example, from dyeing, permanent waves, cleaning of hair with shampoos, and due to environmental pollution. Such hair care products have an influence on the natural structure and properties of hair. For example, the wet and dry combability of hair, the hold and body of hair, and/or protection from increased split ends may be impacted by use of appropriate hair care products.

It has been customary to subject hair to special after-treatments in which the hair is treated with special active ingredients, for example, quaternary ammonium salts or special polymers. This is usually in the form of a rinse. These treatments may result in improved combability, hold, and body of hair while reducing the amount of split ends, depending on the formulation.

Multifunctional cosmetic products are also known in the prior art. In particular, this includes “two-in-one” shampoos, which clean and condition the hair. Such products are appreciated by consumers because the product eliminates the need for at least one procedural step, e.g., conditioning with a traditional hair conditioner.

### DETAILED DESCRIPTION

Similarly, products for altering the natural color of hair play a prominent role in hair cosmetics. Distinctions are made between permanent, semipermanent, and temporary color systems, which are based on chemical and/or natural dyes. Hair colors artificially produced by permanent, semipermanent, or temporary color systems have a drawback, however, in that these hair colors can undergo undesirable changes, e.g., during or after hair cleaning.

“Undesirable changes” refers here to fading or bleeding, as well as the loss of color brilliance of the shade of color of the hair obtained from the respective dyeing. Environmental impacts and/or the effects of the sun can further intensify these changes.

The use of divalent metal salts in hair dye agents to improve the durability and thus fastness of the dyeing is known from EP 2438900 A1, which is incorporated by reference herein.

There is still a need to provide active ingredients and/or combinations of active ingredients for hair treatment agents having favorable, nourishing properties that also strengthen the bonding of dyes to the hair fibers and thus maintain the fastness of artificially-produced hair color, and to further develop hair treatment agents in this regard.

It has, however, been observed that water hardness may have an adverse impact on the care properties of hair treatment agents, such that the level of care provided by a product is either too low or too high, depending on the water quality.

Too high a level of care or “excess care” (the occurrence of a so-called “buildup effect”) of the hair refers primarily to hair that feels greasy, low volume, and/or a stringy, unkempt appearance of the hair, while damaged (cracked, dull, with split ends) hair is an indication of too low a level of care.

The present application therefore addresses the problem of providing favorably skin-compatible and nourishing hair treatment agents that have excellent foaming properties, are readily rinsed with water, and moreover reduce or prevent the washing out of color from dyed hair.

The nourishing hair treatment agents should have a consistent conditioning power, irrespective of the water quality, and, in particular, should not weigh down fine hair and/or damaged hair, but rather structurally strengthen the hair and thus protect against split ends and breakage and improve the combability and feel. The hair treatment agents should also act to reduce germs.

It has now been discovered that a combination of certain ingredients has an especially positive effect on hair treated therewith and on the hair follicles.

A first subject matter of the present invention is hair treatment agents including:
a) at least one anionic surfactant from the group of alkyl sulfates and/or alkyl ether sulfates;
b) at least one amphoteric and/or nonionic surfactant;
c) at least one divalent or trivalent metal salt;
d) at least one cationic polymer; and
e) ethyl lauroyl arginate.

Hair treatment agents in the sense of the present invention include, for example: hair shampoos, hair conditioners, conditioning shampoos, hair sprays, hair rinses, hair cures, hair packings, hair tonics, permanent wave fixative solutions, hair dye shampoos, hair dyeing agents, hair setting formulations, hair styling preparations, blow drying lotions, foam solidifiers, hair gels, hair waxes, and/or combinations thereof. As men are often reluctant to use a plurality of different products and/or to carry out a plurality of application steps, the hair treatment agents according to the present invention are preferably those which men are already using anyway. Preferred agents include shampoos, hair conditioning agents, and/or hair tonics.

The hair treatment agents include at least one anionic surfactant from the group of alkyl sulfates and/or alkyl ether sulfates. Preferred hair treatment agents include, based on the weight of the agent, 0.5 to 20 wt. %, preferably 0.75 to 15 wt. %, further preferably 3 to 12 wt. %, and, in particular, 5 to 10 wt. % anionic surfactant(s) from the group of alkyl sulfates and/or alkyl ether sulfates.

Preferred alkyl sulfates and alkyl polyglycol ether sulfates possess 10 to 18 carbon atoms in the alkyl group, and up to 12 glycol ether groups in the molecule. Particularly preferred anionic surfactants are the alkali or ammonium salts of lauryl ether sulfate with a degree of ethoxylation of 2 to 4 EO.

Especially preferred hair treatment agents include, based on the total weight of the agent, 0.5 to 20 wt. %, preferably 0.75 to 15 wt. %, further preferably 3 to 12 wt. %, and, in particular, 5 to 10 wt. % alkyl(ether) sulfates of the general formula R—(OCH₂—(CH₂)ₙ—OSO₃X, in which R signifies a straight-chain or branched saturated or unsaturated alkyl group having 8 to 24 carbon atoms, n signifies the numbers 0 or 1 to 12, and X signifies an alkali, alkaline earth, ammonium, or alkanolamine ion.

The hair treatment agents according to the present invention include at least one amphoteric surfactant and/or at least one nonionic surfactant. Amphoteric surfactants or zwitterionic surfactants refer to surfactants that have both a negatively-charged functional group and a positively-charged functional group.

Particularly suitable zwitterionic surfactants are the so-called betaines, such as the N-alkyl-N,N-dimethylammonium glycinates, for example cocoalkyl dimethylammonium glycinate; the N-acylaminopropyl-N,N-dimethylammonium glycinates, for example cocoacylaminopropyl dimethylammonium glycinate; and 2-alkyl-3-carboxymethyl-3-hydroxyethyl imidazolines each having 8 to 18 carbon atoms in the alkyl or acyl group, and cocoacylaminoethyl hydroxyethyl carboxymethyl glycinate. A preferred zwitterionic surfactant is the fatty acid amide derivative known by the INCI designation cocamidopropyl betaine.

Further examples of suitable ampholytic surfactants are N-alkylglycines, N-alkylaminopropionic acids, N-alkylaminobutyric acids, N-alkyliminodipropionic acids, N-hydroxyethyl-N-alkyl amidopropyl glycines, N-alkyl taurines, N-alkyl sarcosines, 2-alkylaminopropionic acids, and alkylaminoacetic acids having in each case about 8 to 24 carbon atoms in the alkyl group. Particularly preferred ampholytic surfactants are N-cocoalkyl aminopropionate, cocoacylaminoethyl aminopropionate, and C₁₂-C₁₈ acyl sarcosine.

Preferred hair treatment agents according to the present invention include, based on the weight of the agent, 0.3 to 10 wt. %, preferably 0.5 to 8 wt. %, further preferably 0.75 to 6 wt. %, and, in particular, 1 to 5 wt. % amphoteric surfactant(s).

Particularly preferred hair treatment agents according to the present invention are characterized by including amphoteric surfactant(s) from the groups: N-alkylglycines, N-alkylpropionic acids, N-alkylaminobutyric acids, N-alkyliminodipropionic acids, N-hydroxyethyl-N-alkylamidopropylglycines, N-alkyl taurines, N-alkyl sarcosines, 2-alkylaminopropionic acids having in each case about 8 to 24 carbon atoms in the alkyl group, alkylaminoacetic acids having in each case about 8 to 24 carbon atoms in the alkyl group, N-cocoalkyl aminopropionate, cocoacylaminoethyl aminopropionate, C₁₂-C₁₈ acyl sarcosine, N-alkyl-N,N-dimethylammonium glycinates, for example cocoalkyl dimethylammonium glycinate, N-acylaminopropyl-N,N-dimethylammonium glycinates, for example cocoacylaminopropyl dimethylammonium glycinate, 2-alkyl-3-carboxymethyl-3-hydroxyethyl imidazolines each having 8 to 18 carbon atoms in the alkyl or acyl group, cocoacylaminoethyl hydroxyethyl carboxymethyl glycinate, the compounds known by the INCI designation cocamidopropyl betaine, and the compounds known by the INCI designation disodium cocoamphodiacetate, wherein preferred agents include the amphoteric surfactant(s) in quantities of 0.3 to 10 wt. %, preferably 0.5 to 8 wt. %, further preferably 0.75 to 6 wt. %, and, in particular, 1 to 5 wt. %, based in each case on the total weight of the agent.

Particularly preferred hair treatment agents include, as amphoteric surfactants, betaines of formula (Bet-I):

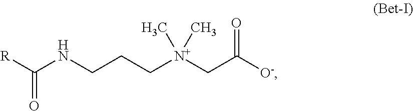

in which R denotes a straight-chain or branched, saturated or mono- or polyunsaturated alkyl or alkenyl residue having 8 to 24 carbon atoms.

These surfactants are referred to according to the INCI nomenclature as amidopropyl betaines, wherein the representatives derived from coconut fatty acids are preferred and referred to as cocamidopropyl betaines. It is particularly preferable according to the present invention to use surfactants of the formula (Bet-I) that are a mixture of the following representatives:

H₃C—(CH₂)₇—C(O)—NH—(CH₂)₃N⁺(CH₃)₂CH₂COO⁻,

H₃C—(CH₂)₉—C(O)—NH—(CH₂)₃N⁺(CH₃)₂CH₂COO⁻,

H₃C—(CH₂)₁₁—C(O)—NH—(CH₂)₃N⁺(CH₃)₂CH₂COO⁻,

H₃C—(CH₂)₁₃—C(O)—NH—(CH₂)₃N⁺(CH₃)₂CH₂COO⁻,

H₃C—(CH₂)₁₅—C(O)—NH—(CH₂)₃N⁺(CH₃)₂CH₂COO⁻, and/or

H₃C—(CH₂)₇—CH═CH—(CH₂)₇—C(O)—NH—(CH₂)₃N⁺(CH₃)₂CH₂COO⁻.

It is particularly preferable to use surfactants of the formula (Bet-I) within narrower quantity ranges. Preferred here are hair treatment agents according to the present invention that, based on the weight of the agent, include 0.25 to 8 wt. %, preferably 0.5 to 7 wt. %, further preferably 0.75 to 6.5 wt. %, and, in particular, 1 to 5.5 wt. % surfactant(s) of the formula (Bet-1).

In addition to the ampho-surfactants of formula (Bet-I), or instead of them, the hair treatment agents according to the present invention may, with particular preference, include as amphoteric surfactants betaines of formula (Bet-II):

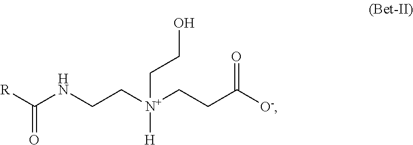

in which R denotes a straight-chain or branched, saturated or mono- or polyunsaturated alkyl or alkenyl residue having 8 to 24 carbon atoms.

These surfactants are referred to according to the INCI nomenclature as amphoacetates, wherein the representatives derived from coconut fatty acids are preferred and referred to as cocoamphoacetates.

For technical reasons relating to manufacture thereof, surfactants of this type always also include betaines of formula (Bet-IIa):

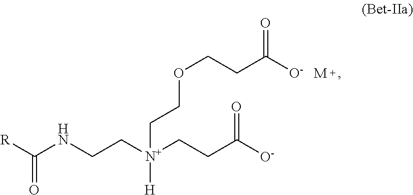

in which R denotes a straight-chain or branched, saturated or mono- or polyunsaturated alkyl or alkenyl residue having 8 to 24 carbon atoms, and M denotes a cation.

These surfactants are referred to according to the INCI nomenclature as amphodiacetates, wherein the representatives derived from coconut fatty acids are preferred and referred to as cocoamphodiacetates.

It is particularly preferable according to the present invention to use surfactants of the formula (Bet-II) that are a mixture of the following representatives:

H₃C—(CH₂)₇—C(O)—NH—(CH₂)₂NH⁺(CH₂CH₂OH)CH₂CH₂COO⁻,

H₃C—(CH₂)₉—C(O)—NH—(CH₂)₂NH⁺(CH₂CH₂OH)CH₂CH₂COO⁻,

H₃C—(CH₂)₁₁—C(O)—NH—(CH₂)₂NH⁺(CH₂CH₂OH)CH₂CH₂COO⁻,

H₃C—(CH₂)₁₃—C(O)—NH—(CH₂)₂NH⁺(CH₂CH₂OH)CH₂CH₂COO⁻,

H₃C—(CH₂)₁₅—C(O)—NH—(CH₂)₂NH⁺(CH₂CH₂OH)CH₂CH₂COO⁻, and/or

H₃C—(CH₂)₇—CH═CH—(CH₂)₇—C(O)—NH—(CH₂)₂NH⁺(CH₂CH₂OH)CH₂CH₂COO⁻.

It is particularly preferable to use surfactants of the formula (Bet-II) within narrower quantity ranges. Preferred here are hair treatment agents according to the present invention that, based on the weight of the agent, include 0.25 to 8 wt. %, preferably 0.5 to 7 wt. %, further preferably 0.75 to 6.5 wt. %, and, in particular, 1 to 5.5 wt. % surfactant(s) of the formula (Bet-II).

In summary, preferred cosmetic agents according to the present invention are those in which the residue R in the formulas (Bet-I) and (Bet-II) is selected from: H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇—, and/or mixtures thereof.

The hair treatment agents may include one or more nonionic surfactant(s). If free of amphoteric surfactants, the hair treatment agents must necessarily include a nonionic surfactant.

Examples of suitable nonionic surfactants include: addition products of 4 to 30 mol of ethylene oxide and/or 0 to 5 mol of propylene oxide to linear fatty alcohols having 8 to 22 carbon atoms, to fatty acids having 12 to 22 carbon atoms, and to alkylphenols having 8 to 15 carbon atoms in the alkyl group; ethylene oxide and polyglycerol addition products to methyl glucoside fatty acid esters, fatty acid alkanolamides, and fatty acid glucamides; C₈-C₃₀ fatty acid monoesters and diesters of addition products of 1 to 30 mol of ethylene oxide to glycerol; amine oxides; sorbitan fatty acid esters and addition products of ethylene oxide to sorbitan fatty acid esters, such as e.g. polysorbates, fatty acid alkanolamides of the following general formula,

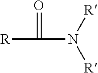

in which R preferably signifies a linear or branched saturated or unsaturated alkyl or alkenyl residue having 8 to 24 carbon atoms, and the residues R′ denote hydrogen or the group —(CH₂)ₙOH, in which n signifies the number 2 or 3, with the proviso that at least one of the residues R′ denotes the aforementioned residue —(CH₂)ₙOH; sugar fatty acid esters and addition products of ethylene oxide to sugar fatty acid esters; addition products of ethylene oxide to fatty acid alkanolamides and fatty amines; alkyl (oligo)glucosides; mixtures of alkyl (oligo)glucosides and fatty alcohols, for example, the commercially available product Montanov® 68; addition products of 5 to 60 mol of ethylene oxide to castor oil and hydrogenated castor oil; partial esters of polyols having 3-6 carbon atoms with saturated fatty acids having 8 to 22 carbon atoms; sterols, wherein sterols are understood to refer to a group of steroids that bear a hydroxy group at the C atom 3 of the steroid structure, and are isolated both from animal tissue (zoosterols) and from vegetable fats (phytosterols), examples of zoosterols include cholesterol and lanosterol. Examples of suitable phytosterols include ergosterol, stigmasterol, and sitosterol. There are also sterols that are isolated from fungi and yeasts (so-called mycosterols); and/or phospholipids, understood to mean principally the glucose phospholipids, which are obtained e.g., as lecithins or phosphatidylcholines from for example, egg yolk or plant seeds (e.g., soybeans).

Suitable alkyl (oligo)glycosides can be selected from compounds of the general formula RO-[G]x, in which [G] is preferably derived from aldoses and/or ketoses having 5-6 carbon atoms, preferably from glucose.

The index number x denotes the degree of oligomerization (DP), i.e. the distribution of mono- and oligoglycosides. The index number x preferably has a value in the range from 1 to 10, more preferably in the range from 1 to 3, wherein it need not be a whole number but may be a fraction which can be determined by analysis.

Particularly preferred alkyl (oligo)glycosides have a degree of oligomerization between 1.2 and 1.5.

The residue R preferably denotes at least one alkyl and/or alkenyl residue having 4 to 24 carbon atoms.

Especially preferred alkyl (oligo)glycosides are compounds that are known under the INCI designations Caprylyl/Capryl Glucoside, Decyl Glucoside, Lauryl Glucoside, and Coco Glucoside.

Suitable amine oxides may be selected from at least one compound of the general formulae (A-I) or (A-II)

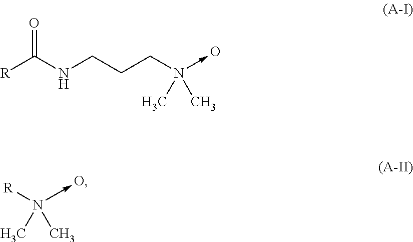

in which R in each case denotes a straight-chain or branched, saturated or mono- or polyunsaturated alkyl or alkenyl residue having 6 to 24 carbon atoms, preferably 8 to 18 carbon atoms.

The surfactants of the aforementioned formulae (A-I) or (A-II) that are known under the INCI designations Cocamine Oxide, Lauramine Oxide, and/or Cocamidopropylamine Oxide and are commercially available from a number of suppliers are preferred in particular.

Suitable C₈-C₃₀ fatty acid monoesters and diesters of addition products of 1 to 30 mol of ethylene oxide to glycerol are preferably understood to be those with the INCI designations PEG(1-10) Glyceryl Cocoate, in particular, PEG-7 Glyceryl Cocoate.

It may also be advantageous to combine the ethoxylated fatty acid esters with other ethoxylated fatty acid esters. Such product mixtures are commercially available, e.g., under the name “Antil 2000®” (INCI designation: PEG-200 Hydrogenated Glyceryl Palmate, PEG-7 Glyceryl Cocoate) from Evonik.

Particularly preferred nonionic surfactants that may be included in the hair treatment agents according to the present invention are: fatty acid alkanolamides, in particular, compounds known by the INCI designations Cocamide MEA and/or Cocamide MIPA; alkyl (oligo)glucosides, in particular, compounds known by the INCI designations Caprylyl/Capryl Glucoside, Decyl Glucoside, Lauryl Glucoside and/or Coco Glucoside; C₈-C₃₀ fatty acid monoesters and diesters of addition products of 1 to 30 mol ethylene oxide to glycerol, in particular, the compound known by the INCI designations PEG-7 Glyceryl Cocoate; and/or addition products of 4 to 30 mol ethylene oxide and/or 0 to 5 mol propylene oxide to linear fatty alcohols having 8 to 22 carbon atoms.

Cocamide MEA and/or PEG-7 Glyceryl Cocoate are especially preferred in light of the foam-stabilizing and moisturizing properties thereof.

Preferred hair treatment agents according to the present invention include, based on the weight of the agent, 0.3 to 10 wt. %, preferably 0.5 to 8 wt. %, further preferably 0.75 to 6 wt. %, and, in particular, 1 to 5 wt. % nonionic surfactant(s).

The hair treatment agents may include (a) cationic surfactant(s). According to the present invention, it is possible to use cationic surfactants of the following types: quaternary ammonium compounds, esterquats, and amidoamines. Preferred quaternary ammonium compounds are ammonium halides, in particular chlorides and bromides, such as alkyl trimethylammonium chlorides, dialkyl dimethylammonium chlorides, and trialkyl methylammonium chlorides. The long alkyl chains of these surfactants preferably have 10 to 18 carbon atoms, such as in, for example, cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride. The preferred cationic surfactants also include the imidazolium compounds known under the INCI designations quaternium-27 and quaternium-83.

Particularly preferred hair treatment agents according to the present invention are characterized by including, as a cationic conditioner, 0.05 to 7.5 wt. %, preferably 0.1 to 5 wt. %, particularly preferably 0.2 to 3.5 wt. %, and, in particular, 0.25 to 2.5 wt. % (based on the total weight of the agent) cationic surfactant(s) from the group of the quaternary ammonium compounds and/or the esterquats and/or the amidoamines, wherein preferred (a) cationic surfactant(s) is/are selected from: alkyl trimethylammonium chlorides having preferably 10 to 18 carbon atoms in the alkyl residue; diallyl dimethylammonium chlorides having preferably 10 to 18 carbon atoms in the alkyl residue; trialkyl methylammonium chlorides having preferably 10 to 18 carbon atoms in the alkyl residue; cetyl trimethylammonium chloride; stearyl trimethylammonium chloride; distearyl dimethylammonium chloride; lauryl dimethylammonium chloride; lauryl dimethyl benzylammonium chloride; tricetyl methylammonium chloride; Quatemium-27; Quatemium-83; N-methyl-N(2-hydroxyethyl)-N,N-(ditalgacyloxyethyl)ammonium methosulfate; N-methyl-N(2-hydroxyethyl)-N,N-(distearoyloxyethyl)ammonium methosulfate; N,N-dimethyl-N,N-distearoyloxyethyl ammonium chloride; and/or N,N-di-(2-hydroxyethyl)-N,N-(fatty acid ester ethyl)-ammonium chloride.

The hair treatment agents include at least one divalent or trivalent metal salt.

To obtain an optimal effect, it is advantageous if the metal salts in the agents according to the present invention are present in dissolved form. In a preferred embodiment, the hair cleaning and care agents according to the present invention therefore include divalent or trivalent metal salts that are water-soluble. “Water-soluble” is understood here to mean being able to completely dissolve at least 1 g of the salt in question in 1 L of water at 20° C.

Suitable divalent or trivalent metal salts may be selected from divalent or trivalent organic and/or inorganic salts.

Particularly suitable cations within these salts may preferably be selected from alkaline earth metal cations as well as from copper, zinc, iron(II), iron(III), and/or aluminum cations.

Alkaline earth metal cations are especially preferred, as are, in particular, calcium and magnesium cations. Particularly suitable organic anions within these salts may preferably be selected from acetate, lactate, succinate, citrate, tartrate, malate, maleate, oxalate, and/or glycolate ions. Acetate, lactate, and/or citrate salts having the aforementioned cations are especially preferred.

Especially preferred organic salts are calcium lactate, calcium citrate, calcium acetate, magnesium lactate, magnesium citrate, and/or magnesium acetate.

Particularly suitable inorganic anions within these salts may be selected from halide, sulfate, phosphate, and/or carbonate ions. Sulfate and/or halide ions, such as chloride and bromide ions, are especially preferred. Especially preferred inorganic salts are calcium chloride, calcium sulfate, magnesium chloride, and/or magnesium sulfate.

The proportion by weight of the at least one divalent or trivalent metal salt to the total weight of the hair treatment agents according to the present invention is preferably 0.01 to 10 wt. %, preferably 0.1 to 7.5 wt. %, further preferably 0.2 to 5 wt. %, and, in particular 0.3 to 3 wt. %.

Hair treatment agents according to the present invention preferably include, based on the weight of the agent, 0.01 to 10 wt. %, preferably 0.1 to 7.5 wt. %, further preferably 0.2 to 5 wt. %, and, in particular, 0.3 to 3 wt. % at least one divalent or trivalent metal salt from the group of the organic or inorganic copper, zinc, iron(II), calcium, magnesium, iron(III), and/or aluminum salts.

Within this embodiment, water-soluble salts are particularly preferred. Especially preferred within this embodiment are calcium lactate, calcium citrate, calcium acetate, magnesium lactate, magnesium citrate, magnesium acetate, calcium halides, calcium hydroxide, magnesium halides, and/or magnesium hydroxide.

The agents according to the present invention include at least one cationic polymer.

Irrespective of which cationic polymer(s) is/are used, preferred hair treatment agents include, based on the total weight of the agent, 0.01 to 3 wt. %, preferably 0.05 to 2 wt. %, further preferably 0.1 to 1.5 wt. %, and, in particular, 0.15 to 0.8 wt. % cationic polymer(s).

Cationic polymers that can preferably be used according to the present invention are described below:

Homopolymers of the general formula (G1-I),

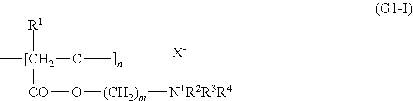

in which R¹ is =—H or —CH₃, and R², R³, and R⁴ independently of each other are selected from C₁₋₄ alkyl, alkenyl, or hydroxyalkyl groups, m=1, 2, 3, or 4, n is a natural number and X⁻ is a physiologically acceptable organic or inorganic anion, and copolymers consisting essentially of the monomer units listed in formula (G1-I), and non-ionogenic monomer units, are particularly preferred cationic polymers. Within the framework of these polymers, those for which at least one of the following conditions applies are preferred according to the present invention: R¹ denotes a methyl group; R², R³, and R⁴ denote methyl groups; and/or m has the value 2.

Appropriate physiologically acceptable counterions X are, for example, halide ions, sulfate ions, phosphate ions, methosulfate ions, and organic ions such as lactate, citrate, tartrate, and acetate ions. Halide ions, in particular chloride, are preferred.

A particularly suitable homopolymer is the poly(methacryloxyethyltrimethylammonium) chloride (crosslinked, if desired) having the INCI name Polyquaternium-37. Such products are commercially available, for example, under the designations Rheocare® CTH (Cosmetic Rheologies) and Synthalen® CR (Ethnichem). The crosslinking may be accomplished, if desired, with the aid of olefinically polyunsaturated compounds, for example divinylbenzene, tetraallyloxyethane, methylene bisacrylamide, diallyl ether, polyallylpolyglyceryl ether, or allyl ethers of sugars or sugar derivatives such as erythritol, pentaerythritol, arabitol, mannitol, sorbitol, sucrose, or glucose. Methylene bisacrylamide is a preferred crosslinking agent.

The homopolymer is preferably used in the form of a non-aqueous polymer dispersion that should include a polymer proportion not less than 30 wt. %. Such polymer dispersions are obtainable commercially under the designations Salcare® SC 95 polymer dispersion (approx. 50% polymer proportion, further components: mineral oil (INCI designation: Mineral Oil) and tridecylpolyoxypropylenepolyoxyethylene ether (INCI designation: P PG-1-Trideceth-6)), and Salcare® SC 96 (approx. 50% polymer proportion, further components: mixture of diesters of propylene glycol with a mixture of caprylic and capric acid (INCI designation: Propylene Glycol Dicaprylate/Dicaprate) and tridecylpolyoxypropylenepolyoxyethylene ether (INCI designation: PPG-1-Trideceth-6)).

Copolymers having monomer units according to formula (G1-l) preferably include acrylamide, methacrylamide, acrylic acid C₁₋₄ alkyl esters, and methacrylic acid C₁₋₄ alkyl esters as non-ionogenic monomer units. Among these non-ionogenic monomers, acrylamide is particularly preferred. As in the case of the homopolymers described above, these copolymers may also be crosslinked. A copolymer preferred according to the present invention is the crosslinked acrylamide-methacryloyloxyethyltrimethylammonium chloride copolymer. Such copolymers, in which the monomers are present at a weight ratio of approximately 20:80, are obtainable commercially as an approximately 50% non-aqueous polymer dispersion under the name Salcare® SC 92.

Further preferred cationic polymers include, for example: quaternized cellulose derivatives such as those commercially obtainable under the designations Celquat® and Polymer JR®; the compounds Celquat® H 100, Celquat® L 200, and Polymer JR® 400 are preferred quaternized cellulose derivatives; cationic alkylpolyglycosides; cationized honey, for example the commercial product Honeyquat® 50; cationic guar derivatives, such as in particular the products marketed under the trade names Cosmedia® Guar and Jaguar®; polymeric dimethyldiallylammonium salts and copolymers thereof with esters and amides of acrylic acid and methacrylic acid, the products obtainable commercially under the designations Merquat® 100 (poly(dimethyldiallylammonium chloride)) and Merquat® 550 (dimethyldiallylammonium chloride/acrylamide copolymer) are examples of such cationic polymers; copolymers of vinylpyrrolidone with quaternized derivatives of dialkylaminoalkyl acrylate and methacrylate, such as with diethylsulfate quaternized vinylpyrrolidone-dimethylaminoethyl methacrylate copolymers. Such compounds are commercially available under the designations Gafquat®734 and Gafquat®755; vinylpyrrolidone-vinylimidazolium methochloride copolymers, such as offered under the designations Luviquat® FC 370, FC 550, FC 905, and HM 552; quaternized poly(vinylalcohol); and/or the polymers known under the names Polyquaternium-2, Polyquaternium-17, Polyquaternium-18, and Polyquaternium-27, having quaternary nitrogen atoms in the main polymer chain.

The polymers known under the designations Polyquaternium-24 (commercial product, e.g. Quatrisoft® LM 200) may also be used as cationic polymers. It is also possible, according to the present invention, to use the copolymers of vinylpyrrolidone, such as are available as commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Cationic protein hydrolysates may also be used as cationic polymers, wherein preferred agents include one or more cationic protein hydrolysates from the group Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, and Quaternium-79 Hydrolyzed Wheat Protein.

It is especially preferable according to the present invention to use cationic polysaccharide polymers as the cationic polymers.

Cationic polysaccharide polymers increase the nourishing performance of the hair treatment agents according to the present invention (in particular, the effectiveness of the agents according to the present invention against hair breakage). Suitable cationic polysaccharide polymers may be selected from cationic cellulose compounds and/or cationic guar derivatives.

Especially preferred hair treatment agents according to the present invention include, as cationic polysaccharide polymer(s), 0.01 to 3 wt. %, preferably 0.05 to 2 wt. %, further preferably 0.1 to 1.5 wt. %, and, in particular, 0.15 to 0.8 wt. % at least one polymer from the group of cationic cellulose polymers and/or cationic guar derivatives, based on the total weight of the agent.

Cationic cellulose compounds in the sense of the present invention are those that bear more than one permanent cationic charge in at least one side chain. Cellulose is composed of beta-1,4-glycosidically linked D-glucopyranose units, and forms unbranched, water-insoluble chains. The side chain of a cellulose is defined as chemical substituents that bond to the cellulose backbone and which are not found in native cellulose, because they have been subsequently introduced e.g. by chemical synthesis.

It is preferred to use quaternized cellulose polymers derived from hydroxy (C₂-C₄) alkyl celluloses, especially preferably from hydroxyethyl celluloses.

Such polymers are known to a person skilled in the art and commercially available from different companies. The cationic cellulose derivatives known under the INCI designations Polyquaternium-4, Polyquaternium-10, Polyquaternium-24, Polyquaternium-67 and/or Polyquaternium-72 are especially preferred. Polyquaternium-10, Polyquaternium-24 and/or Polyquaternium-67 are particularly preferred, especially Polyquaternium-10.

Preferred hair treatment agents according to the present invention include, as cationic polysaccharide polymer(s), 0.01 to 3 wt. %, preferably 0.05 to 2 wt. %, further preferably 0.1 to 1.5 wt. %, and, in particular, 0.15 to 0.8 wt. % at least one polymer from the group of Polyquaternium-4, Polyquaternium-10, Polyquaternium-24, Polyquaternium-67, and/or Polyquaternium-72

Especially preferred hair treatment agents according to the present invention include, based on the total weight of the agent, 0.01 to 3 wt. %, preferably 0.05 to 2 wt. %, further preferably 0.1 to 1.5 wt. %, and, in particular, 0.15 to 0.8 wt. % Polyquaternium-10 as the cationic polysaccharide(s).

Suitable cationic guar derivatives in the sense of the present invention are cationic hydroxyalkyl guar derivatives, preferably cationic hydroxyethyl trimethylammonium guar and/or cationic hydroxypropyl trimethylammonium guar having a mean molecular weight between 100,000 and 2,000,000 daltons. Particularly preferred are the cationic guar polymers that are known under the INCI designation Guar Hydroxypropyltrimonium Chloride and have a molecular weight (weight-average) between 200,000 and 1,600,000 daltons. The cationic charge density of these guar polymers is preferably at least 0.4 meq/g, preferably at least 0.5 meq/g, and, in particular, at least 0.6 meq/g. The nitrogen content thereof is preferably in the range of 1.1 to 1.8 wt. % (based on the total weight thereof).

Cationic guar derivatives known under the INCI designation Guar Hydroxypropyltrimonium Chloride are known to a person skilled in the art and are available, for example, under the trade names Cosmedia® Guar, N-Hance®, and/or Jaguar® from a variety of providers.

Especially preferred hair treatment agents according to the present invention include, based on the total weight of the agent, 0.01 to 3 wt. %, preferably 0.05 to 2 wt. %, further preferably 0.1 to 1.5 wt. %, and, in particular, 0.15 to 0.8 wt. % Guar Hydroxypropyltrimonium as the cationic polysaccharide(s).

The agents according to the present invention include ethyl lauroyl arginate (ELA or LAE)

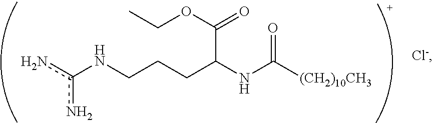

which is also known as ethyl-N^{alpha}-dodecanoyl L-arginate hydrochloride. LAE is commercially available and is considered safe, even for use in food products.

Preferred hair treatment agents according to the present invention include, based on the weight of the agent, 0.001 to 2 wt. %, preferably 0.01 to 1 wt. %, further preferably 0.025 to 0.6 wt. %, and, in particular, 0.05 to 0.4 wt. % ethyl lauroyl arginate HCl.

The hair treatment agents preferably include the aforementioned substances in a cosmetically acceptable carrier. Within the context of the present invention, this preferably is understood to be an aqueous or aqueous-alcoholic carrier.

The cosmetic carrier preferably includes at least 50 wt. %, more preferably at least 60 wt. %, especially preferably at least 70%, and particularly preferably at least 75 wt. % water. The cosmetic carrier may also include 0.01 to 40 wt. %, preferably 0.05 to 30 wt. %, and, in particular, 0.1 to 20 wt. % at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycolene, sorbitol, sorbitan, benzyl alcohol, and/or mixtures of these alcohols.

Water-soluble alcohols are especially preferred. Ethanol, 1,2-propylene glycol, glycerol, benzyl alcohol, and mixtures of these alcohols are particularly preferred.

For the hair treatment agents according to the present invention to have very variable (scalp) skin compatibility, it is advantageous for the agents to have a slightly acidic pH value. It has been discovered that the agents according to the present invention have an especially favorable skin compatibility and mildness in a pH range of 4.2 to 5.8.

In one example, the hair treatment agents according to the present invention therefore preferably have a pH value in the range of 4.2 to 5.8, more preferably 4.25 to 5.6, especially preferably 4.3 to 5.5, extremely preferably 4.35 to 5.4, and particularly preferably 4.4 to 5.3.

The hair treatment agents according to the present invention may include vegetable oils, vegetable butters, and/or waxes. These vegetable oil components endow the hair with an improved combability and manageability, and increase hair shine.

Suitable vegetable oil components include natural (vegetable) oils and/or butters that typically include triglycerides and mixtures of triglycerides.

Preferred natural oils are coconut oil, (sweet) almond oil, walnut oil, peach kernel oil, apricot kemel oil, argan oil, avocado oil, tea tree oil, soybean oil, sesame oil, sunflower oil, Camellia japonica oil, evening primrose oil, rice bran oil, palm kernel oil, mango kernel oil, marula oil, meadowfoam seed oil, safflower oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Beeswax and/or candelilla wax may preferably be used as suitable natural or vegetable waxes.

Particularly preferably vegetable oil components are (sweet) almond oil, peach kernel oil, apricot kernel oil, amaranth seed oil, argan oil, olive oil, jojoba oil, cocoa butter, and/or shea butter. Apricot kernel oil, argan oil, olive oil, and/or jojoba oil are especially preferable

In a preferred embodiment, the hair treatment agents according to the present invention preferably include coconut oil, (sweet) almond oil, walnut oil, peach kemel oil, apricot kemel oil, argan oil, avocado oil, tea tree oil, soybean oil, sesame oil, sunflower oil, Camellia japonica oil, evening primrose oil, rice bran oil, palm kernel oil, mango kernel oil, marula oil, meadowfoam seed oil, safflower oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Within this embodiment, it is especially preferred if the hair treatment agents according to the present invention include (sweet) almond oil, peach kemel oil, apricot kemel oil, amaranth seed oil, argan oil, olive oil, jojoba oil, cocoa butter, and/or shea butter.

The proportion by weight of the at least one vegetable oil, vegetable butter, and/or vegetable wax to the total weight of the hair treatment agents according to the present invention is preferably 0.02 to 2.50 wt. %, more preferably 0.03 to 2.00 wt. %, further preferably 0.04 to 1.50 wt. %, and, in particular, 0.05 to 1.00 wt. %.

In addition to the aforementioned essential and optional components, the hair treatment agents according to the present invention may, in another preferred embodiment for further increasing the nourishing properties of the agents, include at least one additional hair-conditioning ingredient, which may be selected from: protein hydrolysates, vitamins, plant extracts, and/or glycerol.

Suitable protein hydrolysates are understood to be product mixtures that can be obtained by acidically, basically, or enzymatically catalyzed breakdown of proteins.

Protein hydrolysates of plant, animal, and/or marine origin can be used.

Animal protein hydrolysates are, for example, elastin, collagen, keratin, silk, and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Protein hydrolysates of plant origin, for example, soy, almond, rice, pea, potato, and wheat protein hydrolysates, are preferred. Such products are available, for example, under the trademarks Gluadin® (Cognis), DiaMin® (Diamalt), Lexein® (Inolex) and Crotein® (Croda). Cationized protein hydrolysates can also be used, wherein the underlying protein hydrolysate can derive from: animal sources, for example from collagen, milk, or keratin from plant sources, for example from wheat, maize, rice, potatoes, soy, or almonds; from marine life forms, for example from fish collagen or algae; or from protein hydrolysates obtained by biotechnology. The protein hydrolysates underlying the cationic derivatives can be obtained from the corresponding proteins by means of a chemical, in particular alkaline or acid hydrolysis, an enzymatic hydrolysis, and/or a combination of both types of hydrolysis. The hydrolysis of proteins generally gives rise to a protein hydrolysate having a molecular weight distribution from approximately 100 daltons to up to several thousand daltons. Preferred cationic protein hydrolysates are those having an underlying protein component that has a molecular weight of 100 to up to 25,000 daltons, preferably 250 to 5000 daltons. Cationic protein hydrolysates are moreover understood to include quaternized amino acids and mixtures thereof. The quaternization of the protein hydrolysates or the amino acids is frequently performed using quaternary ammonium salts such as for example N,N-dimethyl-N-(n-alkyl)-N-(2-hydroxy-3-chloro-n-propyl) ammonium halides. The cationic protein hydrolysates can moreover also be further derivatized. Typical examples of the cationic protein hydrolysates and derivatives are the commercially available products known under the following INCI designations: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The proportion by weight of the protein hydrolysate(s) to the total weight of the hair treatment agents is preferably 0.01 to 5 wt. %, preferably 0.025 to 3 wt. %, and, in particular 0.05 to 2 wt. %.

Regardless of the source (plant, animal, marine, etc.), protein hydrolysates include individual amino acids, oligopeptides, and optionally polypeptides, depending on the degree of hydrolysis.

Particularly preferably, the hair treatment agents according to the present invention include at least one oligopeptide that includes at least one amino acid sequence Glu-Glu-Glu

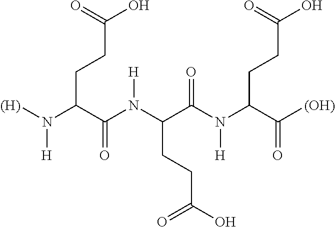

wherein the amino group may be present in free or protonated form and the carboxy groups may be present in free or deprotonated form.

Preferred hair treatment agents according to the present invention are characterized by including, based on the total weight of the agent, 0.0001 to 10 wt. % at least one oligopeptide that includes at least one amino acid sequence Glu-Glu-Glu

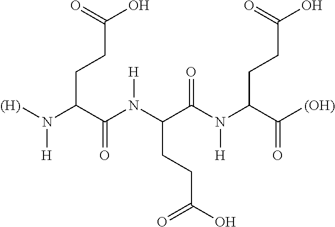

wherein the amino group may be present in free or protonated form and the carboxy groups may be present in free or deprotonated form.

In this, as in all of the formulae below, the bracketed hydrogen atom (H) of the amino group, like the bracketed hydroxy group (OH) of the acid function, indicate that the groups concerned may be present as such (in which case it is an oligopeptide with the respective number of amino acids as illustrated (in formula 3 above)) or that the amino acid sequence is present in an oligopeptide which also includes other amino acids—depending on where the other amino acid(s) is/are bound, the bracketed components in the above formula are replaced by the other amino acid residue(s).

These preferred hair treatment agents according to the present invention include, based on the total weight of the agent, 0.0001 to 10 wt. % at least one oligopeptide that includes at least one amino acid sequence Glu-Glu-Glu, i.e., at least three consecutive glutamic acids.

Oligopeptides within the meaning of the present application are condensation products of amino acids linked by peptide bonds in the manner of an acid amide, with at least three and no more than 25 amino acids.

In preferred hair treatment agents according to the present invention, the oligopeptide has five to 15 amino acids, preferably six to 13 amino acids, particularly preferably seven to 12 amino acids, and, in particular, eight, nine, or 10 amino acids.

Depending on whether other amino acids are bound to the sequence Glu-Glu-Glu and on the nature of these amino acids, the molar mass of the oligopeptide included in the agents according to the present invention may vary. Preferred hair treatment agents according to the present invention are characterized in that the oligopeptide has a molar mass of 650 to 3000 daltons (Da), preferably 750 to 2500 Da, particularly preferably 850 to 2000 Da, and, in particular, 1000 to 1600 Da.

In summary, preferred hair treatment agents are characterized in that the oligopeptide with 5 to 15 amino acids, preferably 6 to 13 amino acids, especially preferably 7 to 12 amino acids, and, in particular, eight, nine, or 10 amino acids, and has a molar mass of 650 to 3000 Da, preferably 750 to 2500 Da, especially preferably 850 to 2000, and, in particular, 1000 to 1600 Da.

As evidenced by the preferred number of amino acids in the oligopeptides and the preferred molar mass range, it is preferable to use oligopeptides that are composed not solely of the three glutamic acids, but also of other amino acids bonded to this sequence. These other amino acids are preferably selected from certain amino acids, whereas certain other representatives are less preferred according to the present invention.

Thus, it is preferable for the oligopeptides used in the agents according to the present invention to not include methionine. It is further preferable for the oligopeptides used in the agents according to the present invention to not include cysteine and/or cystine. It is further preferable for the oligopeptides used in the agents according to the present invention to not include aspartic acid and/or asparagine. It is further preferable for the oligopeptides used in the agents according to the present invention to not include serine or threonine.

On the other hand, it is preferable for the oligopeptides used in the agents according to the present invention to include tyrosine. It is further preferable for the oligopeptides used in the agents according to the present invention to include leucine. It is further preferable for the oligopeptides used in the agents according to the present invention to include isoleucine. It is further preferable for the oligopeptides used in the agents according to the present invention to include arginine. It is further preferable for the oligopeptides used in the agents according to the present invention to include valine.

Especially preferred oligopeptides or amino acid sequences included in the preferred oligopeptides are described below:

A particularly preferred oligopeptide additionally includes tyrosine, which is preferably bound by the acid function thereof to the Glu-Glu-Glu sequence. Preferred hair treatment agents according to the present invention are therefore characterized in that the oligopeptide included therein includes at least one amino acid sequence Tyr-Glu-Glu-Glu

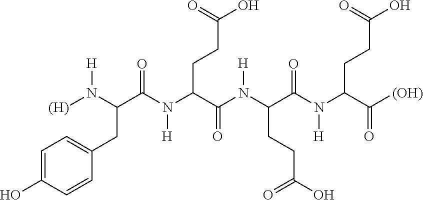

wherein the amino group may be present in free or protonated form and the carboxy groups may be present in free or deprotonated form.

Another particularly preferred oligopeptide additionally includes isoleucine, which is preferably bound by the amino function thereof to the Glu-Glu-Glu sequence. Preferred hair treatment agents according to the present invention are therefore characterized in that the oligopeptide included therein includes at least one amino acid sequence Glu-Glu-Glu-Ile

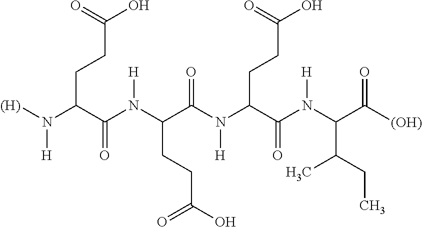

wherein the amino group may be present in free or protonated form and the carboxy groups may be present in free or deprotonated form.

Oligopeptides with both of the above-mentioned amino acids (tyrosine and isoleucine) are preferred according to the present invention. Particularly preferred here are hair treatment agents according to the present invention in which the oligopeptide included in the hair treatment agent includes at least one amino acid sequence Tyr-Glu-Glu-Glu-Ile

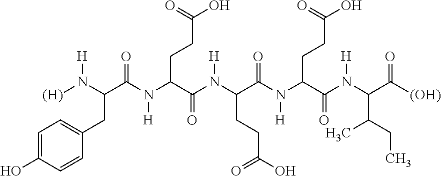

wherein the amino group may be present in free or protonated form and the carboxy groups may be present in free or deprotonated form.

More preferred oligopeptides additionally include arginine, which is preferably present bound to isoleucine. Particularly preferred here are hair treatment agents according to the present invention in which the oligopeptide included in the hair treatment agent includes at least one amino acid sequence Tyr-Glu-Glu-Glu-Ile-Arg

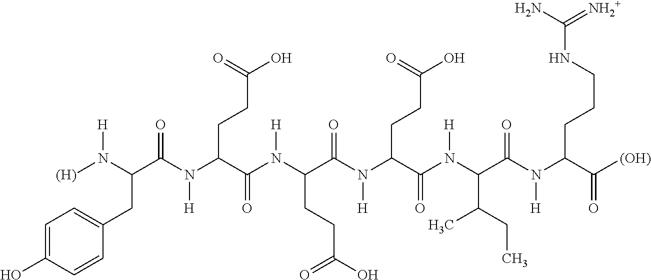

wherein the amino groups may be present in free or protonated form and the carboxy groups may be present in free or deprotonated form.

Even more preferred oligopeptides additionally include valine, which is preferably present bound to the arginine. Further preferred hair treatment agents according to the present invention are therefore characterized in that the oligopeptide included in the hair treatment agent includes at least one amino acid sequence Tyr-Glu-Glu-Glu-Ile-Arg-Val

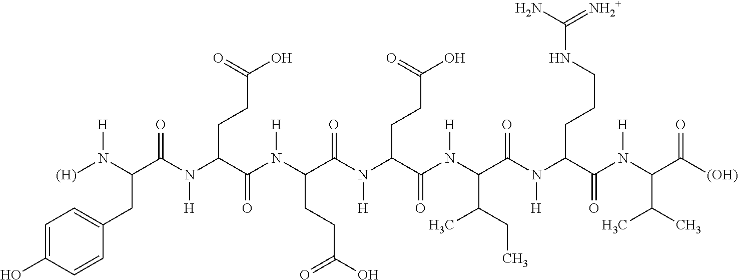

wherein the amino groups may be present in free or protonated form and the carboxy groups may be present in free or deprotonated form.

Even more preferred oligopeptides additionally include leucine, which is preferably present bound to the valine. Further preferred hair treatment agents according to the present invention are therefore characterized in that the oligopeptide included in the hair treatment agent includes at least one amino acid sequence Tyr-Glu-Glu-Glu-Ile-Arg-Val-Leu

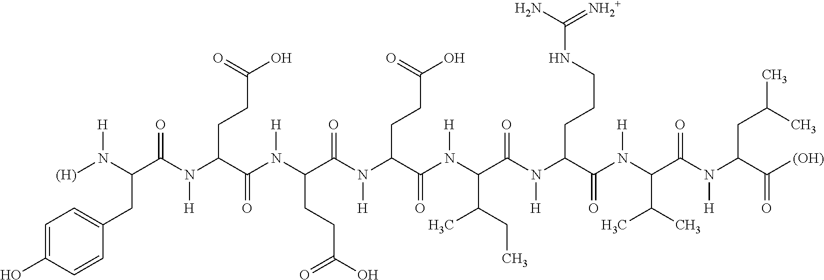

wherein the amino groups may be present in free or protonated form and the carboxy groups may be present in free or deprotonated form.

Especially preferred oligopeptides additionally include leucine, which is preferably present bound to the tyrosine. Further preferred hair treatment agents according to the present invention are therefore characterized in that the oligopeptide included in the hair treatment agent includes at least one amino acid sequence Leu-Tyr-Glu-Glu-Glu-Ile-Arg-Val-Leu

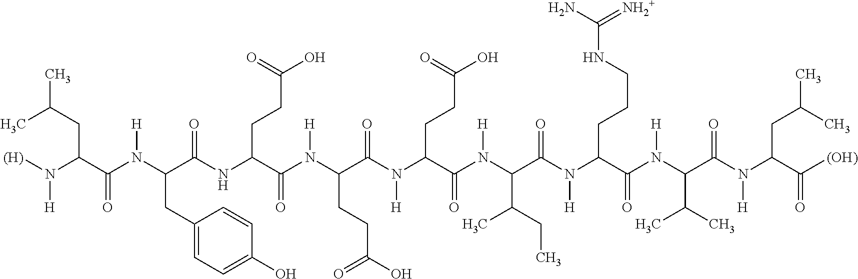

wherein the amino groups may be present in free or protonated form and the carboxy groups may be present in free or deprotonated form.

Very especially preferably, agents according to the present invention include at least two oligopeptides that meet the aforementioned criteria but are different from one another. Thus, for example, it is preferable to use hair treatment agents that include at least two mutually different oligopeptides A and B, which both include the amino acid sequence Glu-Glu-Glu.

Such mutually different oligopeptides A and B are equivalent in bearing three consecutive Glu amino acids in the amino acid sequence thereof, but differ in the amino acids that are bound in front or behind. Mutually different peptides having a partial correspondence, which may be greater than in the three amino acids mentioned above, are preferred.

Thus, further preferred hair treatment agents are characterized in that at least two mutually different oligopeptides A and B both including the amino acid sequence Glu-Glu-Glu-Ile are included in the hair treatment agent.

Also preferred are hair treatment agents which include at least two mutually different oligopeptides A and B that both include the amino acid sequence Tyr-Glu-Glu-Glu.

Still further preferred hair treatment agents are characterized by including at least two mutually different oligopeptides A and B both including the amino acid sequence Glu-Glu-Glu-Ile-Arg.

Also, still further preferred hair treatment agents are characterized in that the hair treatment agent includes at least two mutually different oligopeptides A and B both including the amino acid sequence Tyr-Glu-Glu-Glu-Ile.

Preferred hair treatment agents according to the present invention are therefore characterized in that the oligopeptide includes at least one amino acid sequence Tyr-Glu-Glu-Glu-Ile

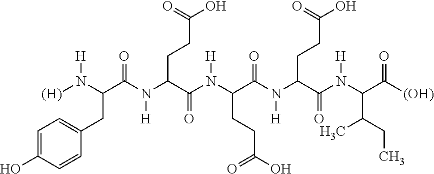

wherein the amino group may be present in free or protonated form and the carboxy groups may be present in free or deprotonated form.

Especially preferred hair treatment agents are characterized in that the hair treatment agent includes at least two mutually different oligopeptides A and B both including the amino acid sequence Glu-Glu-Glu-Ile-Arg.

Also, especially preferred hair treatment agents are characterized in that the hair treatment agent includes at least two mutually different oligopeptides A and B both including the amino acid sequence Tyr-Glu-Glu-Glu-Ile-Arg.

The oligopeptides preferably have an even greater structural correspondence. Thus, hair treatment agents that include at least two mutually different oligopeptides A and B both including the amino acid sequence Glu-Glu-Glu-Ile-Arg-Val are other preferred embodiments of the present invention.

Also preferred embodiments are hair treatment agents which include at least two mutually different oligopeptides A and B that both include the amino acid sequence Tyr-Glu-Glu-Glu-Ile-Arg-Val.

Still further preferred hair treatment agents according to the present invention are characterized by including at least two mutually different oligopeptides A and B both including the amino acid sequence Glu-Glu-Glu-Ile-Arg-Val-Leu.

Also, still further preferred hair treatment agents according to the present invention are characterized by including at least two mutually different oligopeptides A and B both including the amino acid sequence Tyr-Glu-Glu-Glu-Ile-Arg-Val-Leu.

Preferred hair treatment agents according to the present invention are therefore characterized in that the oligopeptide includes at least one amino acid sequence Tyr-Glu-Glu-Glu-Ile-Arg-Val-Leu

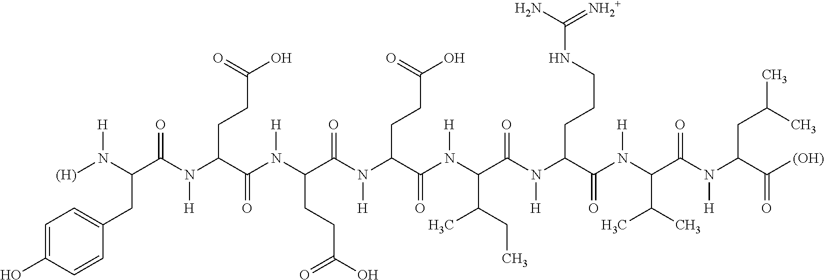

wherein the amino groups may be present in free or protonated form and the carboxy groups may be present in free or deprotonated form,

Especially preferred hair treatment agents according to the present invention are characterized by including at least two mutually different oligopeptides A and B, wherein the oligopeptide A includes the amino acid sequence Leu-Tyr-Glu-Glu-Glu-Ile-Arg-Val-Leu

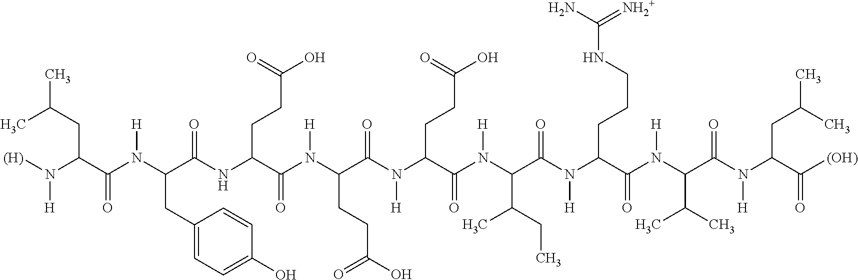

wherein the amino groups may be present in free or protonated form and the carboxy groups may be present in free or deprotonated form and the oligopeptide B includes the amino acid sequence Tyr-Glu-Glu-Glu-Ile-Arg-Val-Leu

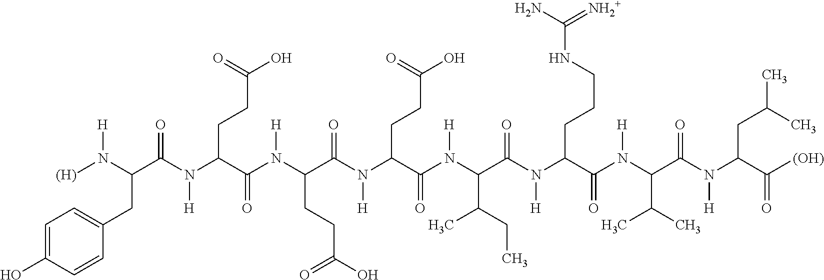

wherein the amino groups may be present in free or protonated form and the carboxy groups may be present in free or deprotonated form.

Especially preferred hair treatment agents of this last-mentioned embodiment include, based on the total weight of the agent, 0.00001 to 1 wt. % oligopeptide A and 0.00001 to 1 wt. % oligopeptide B.

Further preferred hair treatment agents of this last-mentioned embodiment include, based on the total weight of the agent, 0.00005 to 0.1 wt. % oligopeptide A and 0.00005 to 0.1 wt. % oligopeptide B.

Still further preferred hair treatment agents of this last-mentioned embodiment include, based on the total weight of the agent, 0.0001 to 0.01 wt. % oligopeptide A and 0.0001 to 0.001 wt. % oligopeptide B.

The oligopeptides used in the framework of the present invention that meet the aforementioned conditions may advantageously be obtained from keratinous materials. According to the present invention, it is preferred for these oligopeptides to be used in high proportions relative to the total keratinous peptide content of the agents.

It is especially preferred for the highest possible proportion of all of the keratinous peptides included in the agent according to the present invention to meet the aforementioned conditions.

Preferred hair treatment agents according to the present invention are characterized in that at least 0.1 wt. %, preferably at least 0.5 wt. %, particularly preferably at least 1 wt. %, further preferably at least 2.5 wt. %, still further preferably at least 5 wt. %, and, in particular, at least 10 wt. % of all of the keratinous peptides included in the agent include the amino acid sequence Glu-Glu-Glu.

Further preferred hair treatment agents according to the present invention are characterized in that at least 0.1 wt. %, preferably at least 0.5 wt. %, particularly preferably at least 1 wt. %, further preferably at least 2.5 wt. %, still further preferably at least 5 wt. %, and, in particular, at least 10 wt. % of all of the keratinous peptides included in the agent has the amino acid sequence Glu-Glu-Glu-Ile.

Still further preferred hair treatment agents according to the present invention are characterized in that at least 0.1 wt. %, preferably at least 0.5 wt. %, particularly preferably at least 1 wt. %, further preferably at least 2.5 wt. %, still further preferably at least 5 wt. %, and, in particular, at least 10 wt. % of all of the keratinous peptides included in the agent has the amino acid sequence Tyr-Glu-Glu-Glu.

Particularly preferred hair treatment agents according to the present invention are characterized in that at least 0.1 wt. %, preferably at least 0.5 wt. %, particularly preferably at least 1 wt. %, further preferably at least 2.5 wt. %, still further preferably at least 5 wt. %, and, in particular, at least 10 wt. % of all of the keratinous peptides included in the agent has the amino acid sequence Tyr-Glu-Glu-Glu-Ile.

Especially preferred hair treatment agents according to the present invention are characterized in that at least 0.1 wt. %, preferably at least 0.5 wt. %, particularly preferably at least 1 wt. %, further preferably at least 2.5 wt. %, still further preferably at least 5 wt. %, and, in particular, at least 10 wt. % of all of the keratinous peptides included in the agent has the amino acid sequence Tyr-Glu-Glu-Glu-Ile-Arg.

Still further preferred hair treatment agents according to the present invention are characterized in that at least 0.1 wt. %, preferably at least 0.5 wt. %, particularly preferably at least 1 wt. %, further preferably at least 2.5 wt. %, still further preferably at least 5 wt. %, and, in particular, at least 10 wt. % of all of the keratinous peptides included in the agent has the amino acid sequence Tyr-Glu-Glu-Glu-Ile-Arg-Val.

Particularly preferred hair treatment agents according to the present invention are characterized in that at least 0.1 wt. %, preferably at least 0.5 wt. %, particularly preferably at least 1 wt. %, further preferably at least 2.5 wt. %, still further preferably at least 5 wt. %, and, in particular, at least 10 wt. % of all of the keratinous peptides included in the agent has the amino acid sequence Tyr-Glu-Glu-Glu-Ile-Arg-Val-Leu.

The aforementioned conditions relate to the total content of peptides originating from keratinous materials in the agent according to the present invention. In addition to the oligopeptides of keratinous origin, it is also possible, of course, to use other peptides and/or protein hydrolysates, for example, from other native sources. A preferred example is the additional use of wheat protein hydrolysates.

Suitable vitamins are preferably understood to be the following vitamins, provitamins, and vitamin precursors, as well as derivatives thereof:

Vitamin A:

The group of substances referred to as vitamin A includes retinol (vitamin A₁) and 3,4-didehydroretinol (vitamin A₂). β-carotene is the provitamin of retinol. Suitable examples of a vitamin A component according to the present invention are vitamin A acid and esters thereof, vitamin A aldehyde, and vitamin A alcohol and esters thereof, such as palmitate and acetate.

Vitamin B:

The vitamin B group or vitamin B complex includes (inter alia): vitamin B1 (thiamine); vitamin B2 (riboflavin); vitamin B3, encompasses the compounds nicotinic acid and nicotinamide (niacinamide); vitamin B₅ (pantothenic acid and panthenol), within the framework of this group, it is preferable to use panthenol, derivatives of panthenol that can be used are, in particular, the esters and ethers of panthenol, pantolactone, and cationically derivatized panthenols, individual representatives include, for example, panthenol triacetate, panthenol monoethylether, and monoacetate thereof, as well as cationic panthenol derivatives; and/or vitamin B₆ (pyridoxine, pyridoxamine, and pyridoxal).

Vitamin C:

(ascorbic acid): Use in the form of the palmitate, glucosides, or phosphates may be preferred, use in combination with tocopherols may also be preferred.

Vitamin E:

(tocopherols, in particular, α-tocopherol).

Vitamin F:

the term “vitamin F” is generally understood to refer to essential fatty acids, in particular, linoleic acid, linolenic acid, and arachidonic acid.

Vitamin H:

The compound (3aS,4 S,6aR)-2-oxohexahydrothienol[3,4-d]-imidazole-4-valeric acid is referred to as vitamin H, but the common name, biotin, has now become accepted.

Particularly preferred are vitamins, provitamins, and vitamin precursors from the groups A, B, E, and H. Especially preferred are nicotinamide, biotin, pantolactone, and/or panthenol.

The proportion by weight of the vitamin(s), vitamin derivative(s), and/or vitamin precursor(s) to the total weight of the hair treatment agents is preferably 0.001 to 2 wt. %, particularly preferably 0.005 to 1 wt. %, and, in particular, 0.01 to 0.5 wt. %.

Suitable plant extracts are understood to be extracts which can be produced from all parts of a plant. These extracts are conventionally produced by extraction of the entire plant. It can also be preferable in individual cases, however, to produce the extracts exclusively from flowers and/or leaves of the plant. The extracts from green tea, oak bark, stinging nettle, witch hazel, hops, chamomile, burdock, horsetail, whitethom, lime blossom, lychee, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, ginseng, ginger root, Echinacea purpurea, Olea europaea, Boerhavia diffusa root, Foeniculum vulgaris and Apium graveolens are suitable above all.

The extracts of green tea, stinging nettle, witch hazel, chamomile, aloe vera, ginseng, Echinacea purpurea, Olea europaea, and/or Boerhavia diffusa root are particularly preferred for use in the compositions according to the present invention.

Water, alcohols, and mixtures thereof may be used as extracting agents to produce the aforementioned plant extracts. Of the alcohols, low alcohols such as ethanol and isopropanol, but in particular polyhydric alcohols such as ethylene glycol and propylene glycol, are preferred, both as the sole extracting agent and mixed with water. Plant extracts based on water/propylene glycol in the ratio 1:10 to 10:1 have proved to be particularly suitable.

The plant extracts may be used in both pure and diluted form. If used in diluted form, they conventionally include approximately 2 to 80 wt. % of active substance and, as the solvent, the extracting agent or mixture of extracting agents used to obtain them.

The plant extracts may be used in the hair treatment agents according to the present invention (based on the total weight of the agents) preferably in an amount of 0.01 to 10 wt. %, more preferably 0.05 to 7.5 wt. %, and, in particular, 0.1 to 5 wt. %.

Glycerol may be separately added to the hair cleaning and care agents in an amount of up to 10 wt. % (based on the total weight of the agent).

Glycerol may, however, also be a component of the previously-mentioned aqueous-alcoholic carrier.

It has been established that the hair treatment agents according to the present invention are also suitable for use as an anti-dandruff preparation.

The total weight of the anti-dandruff agents to the total weight of the hair treatment agents may preferably be 0.01 to 10 wt. %, more preferably 0.025 to 7.5 wt. %, especially preferably 0.05 to 5 wt. %, and, in particular, 0.075 to 3 wt. %.

Suitable anti-dandruff substances may be selected from piroctone olamine, climbazole, zinc pyrithione, ketoconazoles, salicylic acid, sulfur, selenium sulfide, tar preparations, undecenoic acid derivatives, burdock extracts, poplar extracts, stinging nettle extracts, walnut shell extracts, birch extracts, willow bark extracts, rosemary extracts, and/or amica extracts. Climbazole, zinc pyrithione, and piroctone olamine are preferred.

Examples of further active ingredients, auxiliary substances, and additives that can be included in the hair treatment agents according to the present invention include: humectants; perfumes; UV filters; thickening agents such as gelatins or plant gums, for example agar-agar, guar gum, alginates, xanthan gum, gum arabic, karaya gum, carob seed meal, linseed gums, dextrans, cellulose derivatives, for example methyl cellulose, hydroxyalkyl cellulose, and carboxymethyl cellulose, starch fractions and derivatives such as amylose, amylopectin, and dextrins, clays and phyllosilicates such as, for example, bentonite or fully synthetic hydrocolloids such as, for example, polyvinyl alcohol, the Ca, Mg, or Zn soaps; texturizing agents such as maleic acid and lactic acid; dimethyl isosorbide; cyclodextrins; active ingredients to improve the fiber structure, in particular mono-, di- and oligosaccharides such as, for example, glucose, galactose, fructose, fruit sugar, and lactose; dyes to color the agent; active ingredients such as bisabolol and/or allantoin; complexing agents such as EDTA, NTA, β-alanine diacetic acid, and phosphonic acids; ceramides, understood to be N-acyl sphingosine (fatty acid amides of sphingosine) or synthetic analogs of such lipids (known as pseudoceramides); propellants such as propane-butane mixtures, N₂O, dimethyl ether, CO₂, and air; antioxidants; and/or additional viscosity adjusters such as salts (NaCl).

The agents according to the present invention are preferably so-called rinse-off products, i.e., are rinsed out of the hair after a certain contact time. The contact time preferably amounts to less than one hour, i.e., the consumer preferably does not leave the products in the hair until the next hair wash.

Another subject matter of the present invention is therefore a method for hair treatment, in which an agent according to the present invention is applied to dry or damp hair, left there for a duration of 30 to 300 seconds, and then rinsed out.

The agents according to the present invention lead to a significantly increased strengthening of the inner and outer hair structure. Another subject matter of the present invention is therefore use of agents according to the present invention in order to strengthen the hair structure, in particular, the inner hair structure.

Structural strengthening in the sense of the present invention is understood to be a reduction of damage to keratinous fibers caused by a diverse range of influences. Here, for example, the recovery of the natural firmness plays an essential role. Restructured fibers are characterized, for example, by an improved shine, improved feel, and easier combability. They also have optimized firmness and elasticity. Successful structural strengthening or restructuring may manifest physically as a raise in melting point in comparison to the damaged fibers.

What has been stated regarding the agents according to the present invention also applies, mutatis mutandis, to preferred embodiments of the method according to the present invention and the use according to the present invention.

### Examples

All values represent weight percentage of the agent.

### Series 1 of Hair Shampoos

| | 1-1 | 1-2 | 1-3 | 1-4 | 1-5 | 1-6 |
|---|---|---|---|---|---|---|
| Cocoamidopropyl betaine | 4.0 | 4.0 | 4.0 | 4.0 | 4.0 | 4.0 |
| (AS) | | | | | | |
| Ammonium lauryl sulfate | 9.0 | 9.0 | 9.0 | 9.0 | 9.0 | 9.0 |
| Polyquaternium 10 | 0.3 | 0.3 | 0.3 | 0.3 | 0.3 | 0.3 |
| Cocamide MEA (AS) | 0.8 | 0.8 | 0.8 | 0.8 | 0.8 | 0.8 |
| PEG-7 Glyceryl Cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 1.0 | 1.0 | 1.0 | 1.0 | 1.0 | 1.0 |
| Magnesium sulfate | 0.8 | 0.8 | 0.8 | 0.8 | 0.8 | 0.8 |
| Panthenol | 0.5 | 0.5 | 0.5 | 0.5 | 0.5 | 0.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| Ethyl lauroyl arginate | 0.2 | 0.2 | 0.2 | 0.2 | 0.2 | 0.2 |
| Dimethyloxazolidine | — | 1.3 | 1.3 | 1.3 | 1.3 | 1.3 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-Cymen-5-ol | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | up to 100% |

### Series 2 of Hair Shampoos

| | 2-1 | 2-2 | 2-3 | 2-4 | 2-5 | 2-6 |
|---|---|---|---|---|---|---|
| Sodium laureth sulfate (AS) | 11.0 | 11.0 | 11.0 | 11.0 | 11.0 | 11.0 |
| Coco glucoside (AS) | 4.0 | 4.0 | 4.0 | 4.0 | 4.0 | 4.0 |
| Cocamide MEA (AS) | 0.3 | 0.3 | 0.3 | 0.3 | 0.3 | 0.3 |
| Guar Hydroxypropyl | 0.1 | 0.1 | 0.1 | 0.1 | 0.1 | 0.1 |
| Trimonium Chloride | | | | | | |
| Panthenol | 0.2 | 0.2 | 0.2 | 0.2 | 0.2 | 0.2 |
| Wheat protein hydrolysate | 0.3 | 0.3 | 0.3 | 0.3 | 0.3 | 0.3 |
| Citric acid | 0.4 | 0.4 | 0.4 | 0.4 | 0.4 | 0.4 |
| PEG-120 Methyl Glucose | 0.7 | 0.7 | 0.7 | 0.7 | 0.7 | 0.7 |
| Dioleate | | | | | | |
| Calcium chloride | 0.5 | 0.5 | 0.5 | 0.5 | 0.5 | 0.5 |
| Ethyl lauroyl arginate | 0.4 | 0.4 | 0.4 | 0.4 | 0.4 | 0.4 |
| Dimethyloxazolidine | — | 0.1 | 0.1 | 0.1 | 0.1 | 0.1 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-Cymen-5-ol | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | up to 100% |

What is claimed is:

1. A hair treatment agent, comprising:
at least one anionic surfactant from the group of alkyl sulfates and/or alkyl ether sulfates,
at least one amphoteric and/or nonionic surfactant,
at least one divalent or trivalent metal salt,
at least one cationic polymer, and
ethyl lauroyl arginate.

2. The agent of claim 1, wherein the at least one anionic surfactant from the group of alkyl sulfates and/or alkyl ether sulfates comprises 0.5% to 20% by weight of the agent.

3. The agent of claim 1, wherein the at least one anionic surfactant from the group of alkyl sulfates and/or alkyl ether sulfates comprises 5% to 10% by weight of the agent.

4. The agent of claim 1, wherein the at least one anionic surfactant from the group of alkyl sulfates and/or alkyl ether sulfates comprises alkyl(ether) sulfates of the general formula R—(OCH₂—CH₂)ₙ—OSO₃X, in which R signifies a straight-chain or branched saturated or unsaturated alkyl group having 8 to 24 carbon atoms, n signifies the numbers 0 or 1 to 12, and X signifies an alkali, alkaline earth, ammonium, or alkanolamine ion.

5. The agent of claim 1, wherein the at least one amphoteric and/or nonionic surfactant comprises 0.3% to 10% by weight of the agent.

6. The agent of claim 1, wherein the at least one amphoteric and/or nonionic surfactant comprises 1% to 5% by weight of the agent.

7. The agent of claim 1, wherein the at least one amphoteric and/or nonionic surfactant comprises an amphoteric surfactant.

8. The agent of claim 1, wherein the at least one amphoteric and/or nonionic surfactant comprises 1% to 5% by weight of the agent of a nonionic surfactant.

9. The agent of claim 1, wherein the at least one divalent or trivalent metal salt comprises 0.01% to 10% by weight of the agent.

10. The agent of claim 1, wherein the at least one divalent or trivalent metal salt comprises 0.3% to 3% by weight of the agent.

11. The agent of claim 1, wherein the at least one divalent or trivalent metal salt comprises a copper, zinc, iron(II), calcium, magnesium, iron(III), and/or aluminum salt and/or mixtures thereof.

12. The agent of claim 1, wherein the at least one cationic polymer comprises 0.01% to 3% by weight of the agent.

13. The agent of claim 12, wherein the at least one cationic polymer comprises at least one polymer selected from the group consisting of: cationic cellulose polymers, cationic guar derivatives, and mixtures thereof.

13. The agent of claim 1, wherein the at least one cationic polymer comprises 0.15% to 0.8% by weight of the agent.

14. The agent of claim 1, wherein the agent comprises 0.001% to 2% by weight of ethyl lauroyl arginate HCl.

15. The agent of claim 1, wherein the agent comprises 0.05% to 0.4% by weight of ethyl lauroyl arginate HCl.

16. The agent of claim 1, wherein the agent further comprise two mutually different oligopeptides A and B, which both include the amino acid sequence Glu-Glu-Glu and oligopeptides A and B each comprise 0.00005% to 0.1% by weight of the agent.

17. A hair treatment agent, comprising:
5% to 10% by weight of the agent of at least one anionic surfactant from the group of alkyl sulfates and/or alkyl ether sulfates,
1% to 5% by weight of the agent of at least one amphoteric and/or nonionic surfactant,
0.3% to 3% by weight of the agent of at least one divalent or trivalent metal salt,
0.15% to 0.8% by weight of the agent of at least one cationic polymer, and
0.05% to 0.4% by weight of the agent of ethyl lauroyl arginate.

18. The agent of claim 17, further comprising 0.001% to 10% by weight of the agent glycerol.

19. A method of hair treatment, comprising:
applying a hair treatment agent to hair, the hair treatment agent comprising:
at least one anionic surfactant from the group of alkyl sulfates and/or alkyl ether sulfates,
at least one amphoteric and/or nonionic surfactant,
at least one divalent or trivalent metal salt,
at least one cationic polymer, and
ethyl lauroyl arginate; and
rinsing the hair treatment agent out after 30 sec to 300 sec of contact with the hair.

20. The method of claim 19, wherein the hair treatment agent structurally strengthens both inner and outer hair structures of the contacted hair.
